(19) United States
(12) Patent Application Publication
(10) Pub. No.: US 2021/0401416 A1
(43) Pub. Date: Dec. 30, 2021

(54) PERFORATION SEAL FOR A BLOOD VESSEL

(71) Applicant: Medtronic Vascular, Inc., Santa Rosa, CA (US)

(72) Inventor: Riley J. KING, Santa Rosa, CA (US)

(21) Appl. No.: 16/912,763

(22) Filed: Jun. 26, 2020

(51) Int. Cl. A61B 17/00; A61B 17/12

(52) U.S. Cl. CPC A61B 17/0057; A61B 2017/00871; A61B 2017/00778; A61B 17/12172

(57) ABSTRACT

A system for endovascularly sealing a perforation of a blood vessel is provided. The system includes a seal configured to radially expand from a constricted configuration to an expanded configuration. In embodiments, the seal is biased to expand to the expanded configuration. The seal includes (i) a covered region including a blood-impermeable cover configured to seal the perforation, and (ii) an uncovered region that is located axially adjacent the covered region, does not include the blood-impermeable cover, and is configured to enable distal blood flow through the seal. In some embodiments, the system further includes a cylindrical sheath disposed about the seal in the constricted configuration and configured to slide axially along an outer surface of the seal to enable the seal to expand from the constricted configuration to the expanded configuration.

### TECHNICAL FIELD

The present disclosure relates to a perforation seal or cover for a blood vessel.

### BACKGROUND

The use of percutaneous endovascular procedures has been well established as a minimally invasive technique to deliver a variety of clinical treatments in the vasculature of a patent. Such procedures include, for example, the use of a percutaneous endovascular catheter, which may be used in various applications, including introducing instrumentation into a vein or artery. For example, stent graft delivery, coronary angioplasty, angiography, atherectomy, and deployment, and the like involve accessing and treating the vasculature through a catheter placed in a blood vessel such as the femoral artery.

During or after such treatment, an unwanted perforation of a blood vessel of the patient may occur. This may be caused by a weakened wall of the blood vessel breaking due to the stresses of the treatment. The perforation should be treated immediately to inhibit blood leakage from the vessel.

### SUMMARY

In one embodiment, a system for endovascularly sealing a perforation of a blood vessel is provided. The system includes a seal configured to radially expand from a constricted configuration to an expanded configuration, wherein the seal is biased to expand to the expanded configuration, and wherein the seal includes (i) a covered region including a blood-impermeable cover and (ii) an uncovered region that is located axially adjacent the covered region and does not include the blood-impermeable cover. The system further includes a cylindrical sheath disposed about the seal in the constricted configuration and configured to slide axially along an outer surface of the seal to enable the seal to expand from the constricted configuration to the expanded configuration. The cover is configured to seal the perforation and the uncovered region is configured to enable distal blood flow through the seal when the cylindrical sheath has been slid axially to enable the seal to assume the expanded configuration.

In an embodiment, a seal for sealing a perforation of a blood vessel is provided. The seal includes a basket configured to radially expand to an expanded configuration and radially constrict to a constricted configuration. The basket includes a first uncovered region, a second uncovered region, and a covered region located axially between the first and second uncovered regions. The seal includes a blood-impermeable cover covering the covered region of the basket and located radially outward of at least a portion of the first and second uncovered regions when the basket is in the expanded configuration. When the basket is in the expanded configuration, the cover is configured to seal the perforation and the basket is configured to enable distal blood flow therethrough.

In an embodiment, a method of endovascularly sealing a perforation of a blood vessel is provided. The method includes inserting a lumen and a basket contained therein in a constricted configuration through the blood vessel to a location where the perforation is located. The method also includes retracting the lumen relative to the basket to enable the basket to expand radially outwardly such that a cover of the basket seals the perforation from within the blood vessel while simultaneously enabling distal blood flow through the basket.

### BRIEF DESCRIPTION OF THE DRAWINGS

FIG. 1 is a side view of an endovascular catheter with a seal in a deployed configuration, according to one embodiment.

FIG. 2 is a distal end view of the endovascular catheter of FIG. 1.

FIG. 3 is a proximal-side perspective view of the endovascular catheter of FIG. 1.

FIG. 4 is a distal-side perspective view of the endovascular catheter of FIG. 1.

FIG. 5A is a side view of the endovascular catheter of FIG. 1 in a constricted configuration within a blood vessel having a perforation, according to one embodiment.

FIG. 5B is a side view of the endovascular catheter of FIG. 5A in an expanded configuration within the blood vessel, with the seal sealing the perforation, according to one embodiment.

### DETAILED DESCRIPTION

Embodiments of the present disclosure are described herein. It is to be understood, however, that the disclosed embodiments are merely examples and other embodiments can take various and alternative forms. The figures are not necessarily to scale; some features could be exaggerated or minimized to show details of particular components. Therefore, specific structural and functional details disclosed herein are not to be interpreted as limiting, but merely as a representative basis for teaching one skilled in the art to variously employ the embodiments. As those of ordinary skill in the art will understand, various features illustrated and described with reference to any one of the figures can be combined with features illustrated in one or more other figures to produce embodiments that are not explicitly illustrated or described. The combinations of features illustrated provide representative embodiments for typical applications. Various combinations and modifications of the features consistent with the teachings of this disclosure, however, could be desired for particular applications or implementations.

Directional terms used herein are made with reference to the views and orientations shown in the exemplary figures. A central axis is shown in the figures and described below. Terms such as “outer” and “inner” are relative to the central axis. For example, an “outer” surface means that the surface faces away from the central axis, or is outboard of another “inner” surface. Terms such as “radial,” “diameter,” “circumference,” etc. also are relative to the central axis. The terms “front,” “rear,” “upper” and “lower” designate directions in the drawings to which reference is made.

Unless otherwise indicated, for the delivery system or endovascular catheter with any pre-deployed grafts, the terms “distal” and “proximal” are used in the following description with respect to a position or direction relative to a treating clinician, in which “distal” and “distally” are positions distant from or in a direction away from the clinician, and “proximal” and “proximally” are positions near or in a direction toward the clinician. For a blood vessel or a graft within the vessel (deployed or during deployment), “distal” and “distally” are positions further form the heart by way of blood flow path, and “proximal” and “proximally” are positions nearer the heart by way of blood flow path. These terms are intended for illustrative purposes only, and are not meant to be limiting unless otherwise indicated.

The following detailed description is merely exemplary in nature and is not intended to limit the invention or the application and uses of the invention. Although the description is in the context of treatment of blood vessels such as the aorta, coronary, carotid and renal arteries, the invention may also be used in any other body passageways where it is deemed useful.

Artery perforations are rare but nonetheless feared and sometimes life-threatening complication of percutaneous endovascular procedures. Such perforations may be caused by a breaking or rupture of a weakened wall of the blood vessel due to the stresses of the treatment or contact by surgical tools. The perforation should be treated immediately to inhibit blood leakage from the vessel. One known approach is to introduce an inflatable balloon to the site of the perforation. Once inflated (e.g., with saline), the outer wall of the balloon can expand to contact the surface of the vessel wall and seal the perforation. This measure can provide temporary relief, allowing the surgical technician to assess the perforation and decide a more permanent treatment for the perforation. However, this procedure can take a fair amount of time. For example, full inflation of the balloon can take upwards of 30 seconds in addition to the time needed to track the balloon system to the perforation. A similar duration is once again required when deflating and removing the balloon. During this time, the vessel may continue to leak blood therefrom. Moreover, balloons that are typically used to stabilize perforated vessels will inhibit perfusion distal to the perforation because flow is completely occluded after the balloon is fully inflated. This may lead to ischemia and additional complications.

According to various embodiments described herein, a system for sealing a perforation of a blood vessel is disclosed in which the system includes an endovascular catheter with a constrainable, actuating seal or cover for sealing or covering the perforation while maintaining perfusion therethrough. The system may, for example, include an outer sheath that, when retracted, allows the constrained seal located within the sheath to be exposed and expand. The seal may be hollow or otherwise open to enable proper perfusion (e.g., distal perfusion).

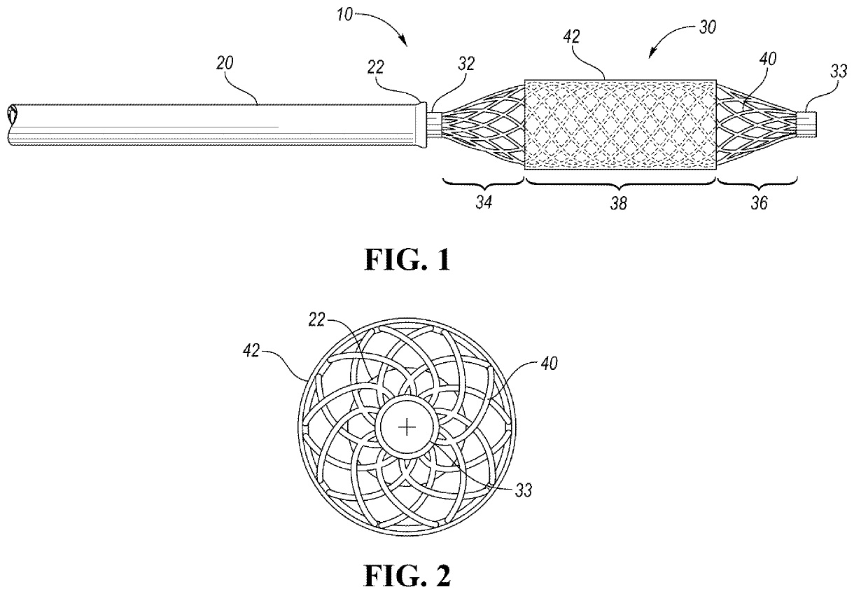

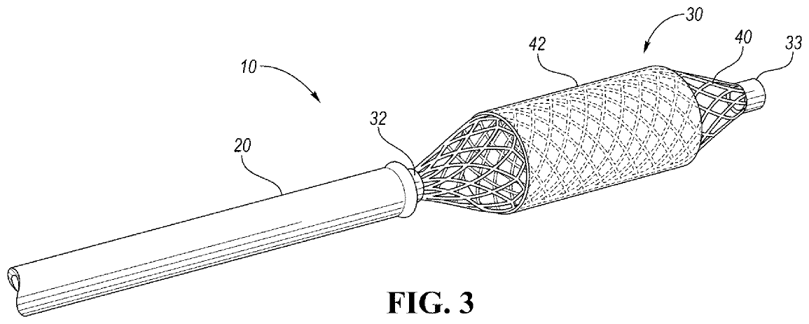

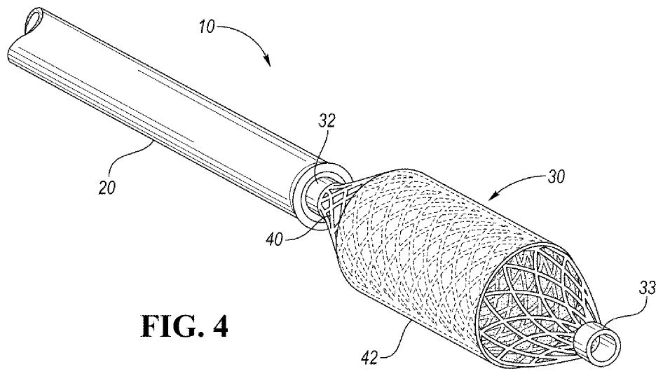

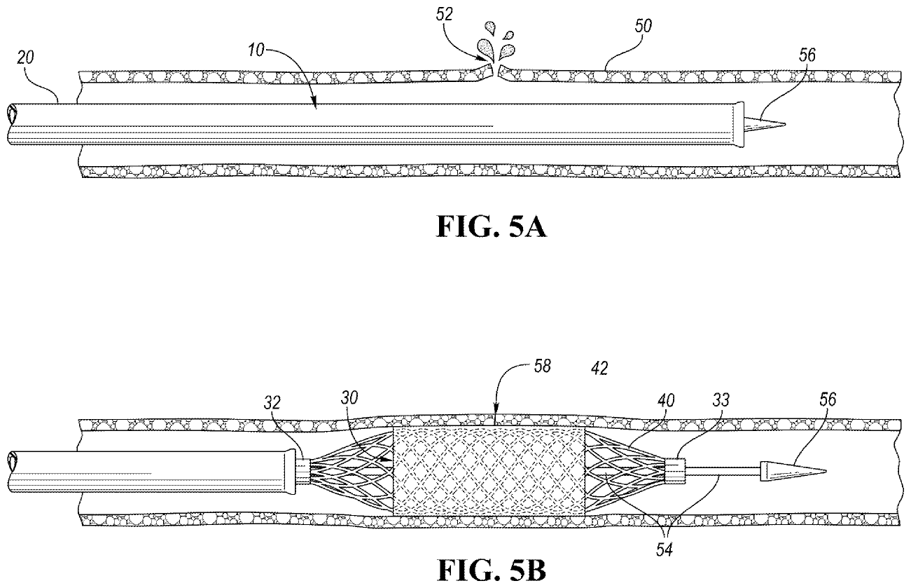

FIG. 1 illustrates a side view of a system 10 for sealing or covering a perforation of a blood vessel is illustrated, with its seal in an expanded configuration. FIG. 2 illustrates a distal end view of the system 10, FIG. 3 illustrates a proximal perspective view of the system 10, and FIG. 4 illustrates a distal perspective view of the system 10. Referring to FIGS. 1-4, the system 10 includes an outer sheath 20, and a seal 30. The outer sheath 20 may be an outer lumen and may be retractable relative to the seal 30. Retraction of the sheath 20 in the proximal direction relative to the seal 30 enables the seal 30 to expand from a constricted or constrained configuration (shown in FIG. 5A, described below) to an expanded or deployed configuration (shown in FIGS. 1-4) in which the seal 30 has expanded in the radial direction.

The sheath 20 extends from a proximal end (not shown) to a distal end 22. Likewise, the seal 30 extends from a proximal end 32 to a distal end 33. When contained within the sheath, the proximal end 32 is located proximally (e.g., toward the surgical technician) relative to the distal ends 22, 32.

The seal 30 has covered regions and uncovered regions. For example, the seal 30 has a first uncovered region 34 located near the proximal end 32 thereof, and a second uncovered region 36 located near the distal end 33 thereof. Between the first uncovered region 34 and the second uncovered region 36 is a covered region 38. The covered region 38 performs the sealing function of the seal 30. The covered region 38 is the region of the seal 30 that is configured to contact the wall of the vessel and seal against the perforation, while the uncovered regions 34, 36 allow lateral or distal perfusion through the seal 30. In other words, the covered region 38 of the seal 30 inhibits undesirable blood flow through the perforation while the uncovered regions 34, 36 enable normal, healthy blood flow through within the confines of the vessel.

While not shown in FIGS. 1-4, the seal 30 may be connected to a middle member extending longitudinally therethrough. For example, the middle member may extend through the proximal end 32, through the uncovered regions 34, 36 and covered region 38, and to the distal end 33. The proximal end 32 and distal end 33 may be fixed about the middle member. The middle member is shown in FIGS. 5A-5B, as described below.

Referring to FIGS. 1-4, the uncovered regions 34, 36 of the seal 30 may comprise a web, mesh, or weave of solid material forming a basket 40. The basket 40 may be made of material identical or similar to stent material in a stent graft (e.g., a self-expanding stent). For example, the basket 40 may be made of a shape-memory material that can flex and bend but return to originally set shape. In other words, the material of the basket 40 may be capable of being constrained in a fully elastic state and without outer diameter loss due to plastic deformation. This material may include or be made of nitinol or a braided wire, for example. In another embodiment, the basket 40 may be made of another bio-compatible and radiopaque material such as cobalt chrome. In such an embodiment, the material of the basket 40 may be formed in such a way that constraining the basket 40 stores elastic energy which is then released (and the shape recovered) once the sheath 20 is retracted to allow the seal 30 to expand. The basket 40 may be manufactured via laser cutting from a solid tube of memory material. Alternatively, the basket 40 may be made via braiding or winding individual strands of memory material. In yet another embodiment, the basket 40 may be manufactured via three-dimensional (3D) printing.

As shown in the illustrated embodiment, the uncovered regions 34, 36 may be tapered. For example, the first uncovered region 34 may be tapered outwardly from the proximal end 32 toward the covered region 38. Likewise, the second uncovered region 36 may be tapered inwardly from the covered region 38 toward the distal end 33. The basket 40 may taper to a connection node at the proximal end 32, and a connection node at the distal end 33. The connection node at the proximal end 32 may be part of an inner lumen that can track along a guidewire for delivery to the perforation. The tapers of the basket 40 allow for a gradual expansion of the seal 30 as the sheath 20 is retracted. For example, as the sheath 20 is moved proximally, the sheath passes over the uncovered region 36, then the cover 42 (described below), and then the uncovered region 34. The seal 30 is allowed to gradually expand radially outwardly as the sheath 20 passes the uncovered region 34. The tapers also facilitate the removal of the seal 30. For example, the seal 30 can be withdrawn by first advancing the sheath 20 distally (forward) across the first uncovered region 34, which compresses the seal 30 within the sheath 20 into the compressed or constrained configuration. Once the seal 30 is compressed within the sheath 20, the system 10 can be removed from the vessel.

The covered region 38, which is configured to perform the sealing function to seal the perforation, can be made of material similar to the graft material of a stent graft. For example, the covered region 38 may include a cylindrically-shaped cover 42 that includes or is made of a nylon or woven material such as polyethylene terephthalate (PET), Dacron, laminated layer of Polytetrafluoroethylene PTFE film, or other similar material impermeable to blood. The cover 42 may be affixed to the material of the basket 40 such that as the basket 40 expands radially, so does the covered region 38. The cover 42 may be affixed to either or both of the inner surface and outer surface of the basket 40. In one embodiment, the cover 42 is fixed to the inner surface of the basket 40 so as to not impair blood flow proximally/distally through the seal 30. This also prevents a prolonged metal-to-blood contact with the blood flowing through the vessel, which may otherwise have a potential to cause clots.

There is no cover 42 covering the uncovered regions 34, 36. This allows blood to flow freely distally to the perforation and through the seal 30 when the seal 30 is fully deployed.

In another embodiment, the cover 42 may be designed so as to retain perfusion to one or more side branch vessels in the region near the perforation by providing fenestrations in the cover at known clocking/orientation. Such fenestrations allow blood to flow freely through them and may be marked by radiopaque material such as gold or platinum thread for high visibility and accurate positioning.

Since the seal 30 is configured to seal the perforation temporarily until a more permanent solution is employed, the cover 42 may be relatively thin. For example, typical graft coverings for permanent implants have thicknesses on the order of 0.001 inch to 0.010 inch, allowing for durability over the course of ten years or more of service without failure due to material fatigue. A temporary sealing devices such as described in some embodiments of the present invention may be manufactured with thicknesses as low as approximately 0.0005 inch since their service life is intended to be only minutes or hours rather than years.

During a surgical procedure such as a percutaneous endovascular procedure, a guidewire may be inserted to a desired treated area of a blood vessel. If a perforation of the treated vessel may inadvertently occur, the surgical technician can utilize the system 10 to at least temporarily treat the perforation. In one use-case example, the surgical technician may remove the devices from the vessel by retracting the devices along the guidewire and out of the patient. The technician may then insert the system over the guidewire and insert percutaneously to the vessel. During insertion of the system, the seal 30 is in the constricted configuration, bound within the sheath 20. A middle member of the system, or the hollow distal end 33 and distal end 22, may track along the guidewire until the system 10 reaches the desired location. Once in position, the technician can retract the sheath 20, allowing the basket to expand. The cover 42 seals against the perforation of the vessel, while blood is still allowed to flow distally within the vessel through the basket 40. The sheath 20 can either be retracted fully out of the patient, or can remain adjacent to the expanded seal 30 until a more permanent solution is derived. When ready, the technician can push the sheath toward the distal end 33, sliding over uncovered region 34 to constrict the seal 30. Alternatively, the seal 30 may be pulled or retracted back into the sheath. Once the seal 30 is again partially or fully constricted within the sheath 20, the system may be removed from the patient by being retracted along the guidewire.

Instead of, or in addition to, the sheath 20, the connection nodes (e.g., where the basket 40 tapers and connects to a lumen feature at the proximal end 32 and distal end 33) may be capable of translating axially relative to one to expand/contract the seal 30. For example, the proximal end 32 may be part of, or connected to, a first lumen, and the distal end 33 may be part of, or connected to, a second lumen. At least one of the first or second lumens may be connected to a push rod or tension wire that runs substantially parallel to the main catheter body. Axial movement of the proximal end 32 and the first lumen away from the distal end 33 can cause the basket 40 to elongate and constrict. Likewise, axial movement of the proximal end 32 and the first lumen toward the distal end 33 can cause the basket 40 to constrict axially but expand radially. The basket 40 may therefore move pursuant to the Poisson effect in which the basket 40 expands in a direction (e.g., radially) that is perpendicular to the direction of compression (e.g., axially), and constricts in a direction (e.g., radially) that is perpendicular to the direction of tension (e.g., axially).

FIGS. 5A-5B illustrate the use of the system 10 within a blood vessel 50. The blood vessel 50 may have an undesirable perforation 52. The perforation 52 may be remote or spaced from the insertion or access site of the system 10. In other words, in at least one embodiment, the system 10 is used to treat a perforation that is not the access site. FIG. 5A shows the system 10 in the constricted configuration with the seal 30 constricted within the interior of the sheath 20, while FIG. 5B shows the system 10 in the expanded configuration with the seal 30 expanded. Referring to FIG. 5A, the system 10 may be inserted into the vessel 50 by tracking along a guidewire (not shown). A middle member 54 (FIG. 5B) may be provided within the system 10 that comprises a hollow lumen to track along the guidewire. The middle member may end with a conical tip 56 that defines a leading edge of the system 10 when tracking along the guidewire.

Once delivered to the desired location within the vessel 50 where the perforation 52 is located, the sheath 20 may be retracted, as shown in FIG. 5B. Retraction of the sheath 20 allows the basket 40 and cover 42 of the seal 30 to expand radially outwardly. The cover 42 seals against the perforation of the vessel 50, thus creating a sealed perforation 58 that inhibits blood from leaking through the perforation. Meanwhile, blood is allowed to flow distally through the basket 40.

In an embodiment described above, the sheath 20 may not be necessary and instead the basket can expand and contract via the Poisson effect. To do so, referring to FIG. 5B, the connection node at the proximal end 32 may be a cylindrical region capable of sliding axially along the middle member 54 relative to the connection node at the distal end 33. In one embodiment, the connection node at the distal end 33 may be fixed to the middle member 54, while the connection node at the proximal end 32 may slide along the middle member 54 to expand and contract the basket 40 as described above.

While embodiments of the system 10 described above may be for temporarily sealing a perforation of a blood vessel until a more permanent solution is derived, in other embodiments the system 10 may be a more permanent fixture for more permanently sealing the perforation. For example, in one embodiment, the seal 30, including the basket 40 and cover 42, is capable of being released from the delivery system upon delivery within the blood vessel. The seal 30 may be released into a substantially cylindrical covered stent capable of remaining at the perforation to provide a long-term therapeutic solution. This would provide the surgical technician with the option of temporarily sealing the perforation, or sealing the perforation with a long-term solution without adding an additional covered stent, coils, etc.

While exemplary embodiments are described above, it is not intended that these embodiments describe all possible forms encompassed by the claims. The words used in the specification are words of description rather than limitation, and it is understood that various changes can be made without departing from the spirit and scope of the disclosure. As previously described, the features of various embodiments can be combined to form further embodiments of the invention that may not be explicitly described or illustrated. While various embodiments could have been described as providing advantages or being preferred over other embodiments or prior art implementations with respect to one or more desired characteristics, those of ordinary skill in the art recognize that one or more features or characteristics can be compromised to achieve desired overall system attributes, which depend on the specific application and implementation. These attributes can include, but are not limited to cost, strength, durability, life cycle cost, marketability, appearance, packaging, size, serviceability, weight, manufacturability, ease of assembly, etc. As such, to the extent any embodiments are described as less desirable than other embodiments or prior art implementations with respect to one or more characteristics, these embodiments are not outside the scope of the disclosure and can be desirable for particular applications.

What is claimed is:

1. A system for endovascularly sealing a perforation of a blood vessel, the system comprising:
a seal configured to radially expand from a constricted configuration to an expanded configuration, wherein the seal is biased to expand to the expanded configuration, and wherein the seal includes (i) a covered region including a blood-impermeable cover and (ii) an uncovered region that is located axially adjacent the covered region and does not include the blood-impermeable cover; and
a cylindrical sheath disposed about the seal in the constricted configuration and configured to slide axially along an outer surface of the seal to enable the seal to expand from the constricted configuration to the expanded configuration;
wherein the cover is configured to seal the perforation and the uncovered region is configured to enable distal blood flow through the seal when the cylindrical sheath has been slid axially to enable the seal to assume the expanded configuration.

2. The system of claim 1, wherein the seal includes a basket of a memory material configured to bend and return to original shape.

3. The system of claim 2, wherein the memory material is formed in a weave, mesh, or web configured to enable blood to flow distally through gaps in the basket.

4. The system of claim 1, wherein the uncovered region is a first uncovered region located distally relative to the covered region, and the seal includes a second uncovered region located proximal relative to the covered region.

5. The system of claim 4, wherein when the seal is in the expanded configuration, the first and second uncovered regions taper radially inwardly away from the covered region.

6. The system, of claim 5, wherein the first uncovered region tapers radially inwardly toward a distal end of the seal, and the second uncovered region tapers radially inwardly toward a proximal end of the seal, wherein the distal end and proximal end are smaller in diameter than the sheath when in the expanded and constricted configurations,
and wherein the covered region is larger in diameter than the sheath when in the expanded configuration and smaller in diameter than the sheath when in the constricted configuration.

7. The system of claim 6, wherein the distal end and proximal end are hollow and configured to endovascularly track along a guidewire.

8. The system of claim 1, wherein the cover comprises at least one of polytetrafluoroethylene (PTFE), polyethylene terephthalate (PET), or Dacron.

9. A seal for sealing a perforation of a blood vessel, the seal comprising:
a basket configured to radially expand to an expanded configuration and radially constrict to a constricted configuration, the basket including a first uncovered region, a second uncovered region, and a covered region located axially between the first and second uncovered regions; and
a blood-impermeable cover covering the covered region of the basket and located radially outward of at least a portion of the first and second uncovered regions when the basket is in the expanded configuration;
wherein when the basket is in the expanded configuration, the cover is configured to seal the perforation and the basket is configured to enable distal blood flow therethrough.

10. The seal of claim 9, further comprising a sheath disposed about the basket and configured to maintain the basket in the constricted configuration, wherein the sheath is configured to slide axially along an outer surface of the basket to enable the basket to expand from the constricted configuration to the expanded configuration.

11. The seal of claim 9, wherein, when the basket is in the expanded configuration, the first uncovered region tapers radially inwardly toward a distal end of the basket and the second uncovered region tapers radially inwardly toward a proximal end of the basket.

12. The seal of claim 11, wherein the distal end is axially moveable relative to the proximal end to transition the basket between the expanded configuration and the constricted configuration.

13. The seal of claim 11, wherein the distal end and the proximal end of the basket have a fixed diameter, and the covered region has an expandable diameter.

14. The seal of claim 9, wherein the basket is made of a memory material configured to bend and return to original shape.

15. A method of endovascularly sealing a perforation of a blood vessel, the method comprising:
inserting a lumen and a basket contained therein in a constricted configuration through the blood vessel to a location where the perforation is located;
retracting the lumen relative to the basket to enable the basket to expand radially outwardly such that a cover of the basket seals the perforation from within the blood vessel while simultaneously enabling distal blood flow through the basket.

16. The method of claim 15, further comprising, subsequent to the step of retracting, advancing the lumen relative to the basket to force the basket to constrict radially inwardly such that the cover is moved radially inwardly from the location where the perforation is located.

17. The method of claim 16, wherein the step of advancing includes advancing the lumen over a tapered region of the basket that tapers radially inwardly toward a distal end thereof.

18. The method of claim 16, further comprising, subsequent to the step of advancing, withdrawing the lumen and basket from the blood vessel.

19. The method of claim 15, wherein the basket includes a first uncovered region, a second uncovered region, and a covered region located between the first and second uncovered regions, wherein the step of retracting the lumen expands the covered region radially outwardly beyond the first and second uncovered regions.

20. The method of claim 19, wherein the step of retracting includes retracting the lumen along a tapered region of the basket such that the basket expands to a degree that corresponds with the retraction of the lumen.
